(19) United States
(12) Patent Application Publication
(10) Pub. No.: US 2018/0271492 A1
(43) Pub. Date: Sep. 27, 2018

(54) ULTRASONIC APPARATUS AND BEAMFORMING METHOD FOR THE SAME

(71) Applicant: SAMSUNG ELECTRONICS CO., LTD., Gyeonggi-do (KR)

(72) Inventors: Tae Ho JEON, Seoul (KR); Jong Keun SONG, Yongin-si (KR)

(73) Assignee: SAMSUNG ELECTRONICS CO., LTD., Suwon-si (KR)

(21) Appl. No.: 15/525,121

(22) PCT Filed: Nov. 7, 2014
(86) PCT No.: PCT/KR2014/010690

§ 371 Date: May 8, 2017

(51) Int. Cl. A61B 8/00; B06B 1/02; G01S 15/89

(52) U.S. Cl. CPC A61B 8/4488; A61B 8/4494; B06B 1/0284; B06B 1/0622; B06B 2201/76; B06B 1/0292; G01S 15/8925

(57) ABSTRACT

There are provided an ultrasonic apparatus and a beamforming method for the ultrasonic apparatus. According to an aspect, an ultrasonic apparatus includes a 2D array transducer in which a plurality of elements are arranged in a plurality of rows and columns; a signal supplier configured to supply an ultrasonic signal to the 2D array transducer; a first delayer configured to delay the ultrasonic signal to output a plurality of first delay signals corresponding to any one of the number of rows and the number of columns; and a second delayer configured to delay one of the plurality of first delay signals to output a plurality of second delay signals corresponding to the other one of the number of rows and the number of columns, and transmit the plurality of second delay signals to the 2D array transducer.

### TECHNICAL FIELD

Embodiments of the present invention relate to an ultrasonic apparatus for imaging an ultrasonic signal and a beamforming method for the same.

### BACKGROUND ART

An ultrasonic diagnostic apparatus is an apparatus that radiates an ultrasound toward a specific region inside a body from a surface of the body of an object and obtains an image of a section of a soft tissue or blood flow using information on a reflected echo ultrasound in a noninvasive manner.

The ultrasonic diagnostic apparatus is advantageous in that it is small, cheap, can display in real time, and has high safety without exposure to X-rays. Due to these advantages, the ultrasonic diagnostic apparatus is being widely used for heart, breast, abdomen, urinary organ, and obstetrics diagnoses.

The ultrasonic diagnostic apparatus emits ultrasonic waves through an ultrasonic probe, which can be classified according to the manner in which the transducer elements are arranged. Recently, research is being conducted on a method of generating an ultrasound image by emitting ultrasounds using a 2D array probe in which elements are arranged in two dimensions.

### DISCLOSURE OF INVENTION

### Technical Problem

The present invention provides an ultrasonic apparatus for beamforming by setting a set of elements belonging to either a column or a row in a 2D array transducer as a sub-array transducer, and a beamforming method for the ultrasonic apparatus.

### Solution to Problem

According to an aspect, an ultrasonic apparatus includes a 2D array transducer in which a plurality of elements are arranged in a plurality of rows and columns; a signal supplier configured to supply an ultrasonic signal to the 2D array transducer; a first delayer configured to delay the ultrasonic signal to output a plurality of first delay signals corresponding to any one of the number of rows and the number of columns; and a second delayer configured to delay one of the plurality of first delay signals to output a plurality of second delay signals corresponding to the other one of the number of rows and the number of columns, and transmit the plurality of second delay signals to the 2D array transducer.

According to another aspect, an ultrasonic apparatus includes a 2D array transducer configured to generate a plurality of ultrasonic echo signals by a plurality of elements arranged in a plurality of columns and rows; a third delayer configured to delay each of the plurality of ultrasonic echo signals generated in each of the plurality of elements, and output a plurality of third delay signals; a first adder configured to combine the third delay signals corresponding to one of the plurality of third delay signals to output a plurality of first combined signals corresponding to one of the number of the rows and the number of the columns; a fourth delayer configured to delay the plurality of first combined signals to output a plurality of fourth delay signals; and a second adder configured to combine the plurality of fourth delay signals to output a second combined signal.

According to an aspect, a beamforming method for an ultrasonic apparatus including a 2D array transducer in which a plurality of elements are arranged in a plurality of columns and rows, the method includes generating an ultrasonic signal; delaying the ultrasonic signal to output a plurality of first delay signals, the number of which corresponds to one of the number of the rows and the number of the columns; delaying one of the plurality of first delay signals to output a plurality of second delay signals, the number of which corresponds to the other one of the number of the rows and the number of the columns; and transmitting the plurality of second delay signals to the 2D array transducer.

According to another aspect, a beamforming method for an ultrasonic apparatus including a 2D array transducer configured to generate a plurality of ultrasonic echo signals by a plurality of elements arranged in a plurality of columns and rows, the method includes delaying each of the plurality of ultrasonic echo signals generated by each of the plurality of elements to output a plurality of third delay signals; combining the third delay signals corresponding to one of the same row and the same column among the plurality of third delay signals to output a plurality of first combined signals corresponding to one of the number of rows and the number of columns; delaying the plurality of first combined signals to output a plurality of fourth delay signals; and summing the plurality of fourth delay signals, and outputting one second combined signal.

### Advantageous Effects of Invention

According to an aspect of the ultrasonic apparatus and the beamforming method for the same, an amount of computation may be reduced in performing beamforming using a 2D array transducer. Also, In implementing a circuit for beamforming, the complexity of the circuit may be reduced.

### BRIEF DESCRIPTION OF DRAWINGS

These and/or other aspects of the invention will become apparent and more readily appreciated from the following description of the embodiments, taken in conjunction with the accompanying drawings of which:

FIG. 1 is a perspective view illustrating an ultrasonic imaging apparatus according to an embodiment;

FIG. 2 is a diagram illustrating the exterior of a two dimensional (2D) array probe according to an embodiment;

FIG. 3 is a block diagram of an ultrasonic apparatus according to an embodiment;

FIG. 4 is a diagram for explaining a method of configuring a sub-array transducer;

FIGS. 5A and 5B are views for explaining a method of configuring a sub-array transducer to perform beamforming in ultrasonic transmission;

FIG. 6 is a view for explaining a structure in which a 2D array transducer, a first delayer and a second delayer are arranged;

FIG. 7 is a block diagram illustrating another embodiment of the ultrasonic apparatus;

FIGS. 8A and 8B are views for explaining a method of configuring a sub-array transducer to perform beamforming upon ultrasonic reception;

FIG. 9 is a view for explaining a structure in which the 2D array transducer, the third delayer, the fourth delayer, the first adder, and the second adder are arranged;

FIG. 10 is a flowchart according to an embodiment of a beamforming method of the ultrasonic apparatus;

FIG. 11 is a flowchart according to another embodiment of the beam forming method of the ultrasonic apparatus.

### BEST MODE FOR CARRYING OUT THE INVENTION

Hereinafter, an ultrasonic apparatus and a beamforming method for the same according to an embodiment will be described in detail with reference to the accompanying drawing.

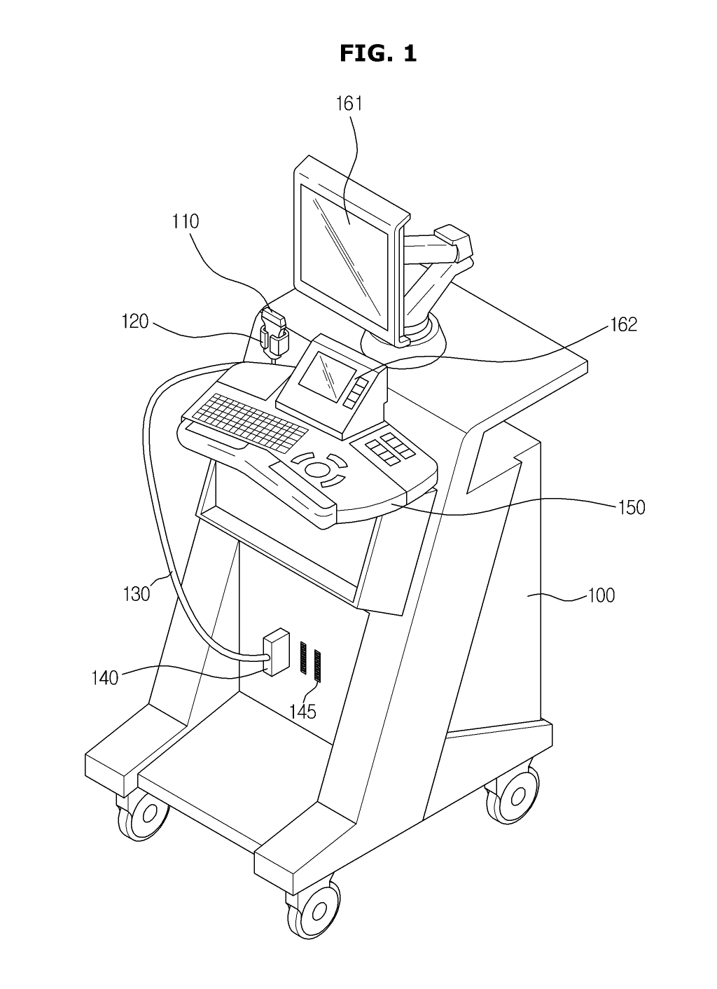

FIG. 1 is a perspective view illustrating an ultrasonic imaging apparatus according to an embodiment. As illustrated in FIG. 1, the ultrasonic imaging apparatus may include a main body 100, an ultrasonic probe 110, an input unit 150, and a display unit 160.

At least one female connector 145 may be provided on a side of the main body 100. A male connector 140 connected to a cable 130 may be physically coupled with the female connector 145.

Meanwhile, a plurality of castors (not illustrated) for moving the ultrasonic imaging apparatus may be provided on the bottom of the main body 100. The plurality of castors may fix the ultrasonic imaging apparatus at a specific place or move it in a specific direction.

The ultrasonic probe 110 is a unit that comes in contact with a surface of a body of an object and may transmit and receive an ultrasound. Specifically, the ultrasonic probe 110 transmits the ultrasound to an inside of the object according to a transmission signal provided from the main body 100, and receives an echo ultrasound reflected from a specific region inside the object and transmits the echo ultrasound to the main body 100. An end of the cable 130 may be connected to the ultrasonic probe 110 and the male connector 140 may be connected to the other end of the cable 130. The male connector 140 connected to the other end of the cable 130 may be physically coupled with the female connector 145 of the main body 100.

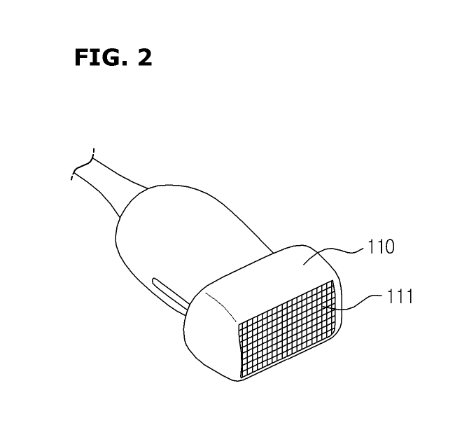

Hereinafter, a two dimensional (2D) array probe will be described as an exemplary ultrasonic probe with reference to FIG. 2. FIG. 2 is a diagram illustrating the exterior of a 2D array probe according to an embodiment.

A kind of the ultrasonic probe may be identified according to a method of arranging transducer elements. A 1D array probe in which elements are one-dimensionally arranged in a surface of the ultrasonic probe includes a linear array probe in which elements are arranged in a straight line, a phased array probe, and a convex array probe in which elements are arranged in a curved line. On the other hand, an ultrasonic probe in which elements are two-dimensionally arranged is referred to as a 2D array probe.

As illustrated in FIG. 2, elements may be two-dimensionally arranged in a surface of a 2D array probe 110. While FIG. 2 exemplifies a case in which elements are arranged on a plane, elements may also be arranged into a curved form on a surface of the 2D array probe 110.

Hereinafter, it is assumed that the ultrasonic probe is a 2D array probe.

Referring again to FIG. 1, the input unit 150 is a unit that may receive a command related to an operation of the ultrasonic imaging apparatus. For example, a command to select a mode, such as an A-mode (amplitude mode), a B-mode (brightness mode), and an M-mode (motion mode), or an ultrasound diagnosis starting command may be received. The command input through the input unit 150 may be transmitted to the main body 100 via wired and/or wireless communication.

The input unit 150 may include at least one of, for example, a keyboard, a foot switch, and a foot pedal. The keyboard may be implemented in hardware and located on the top of the main body 100. This keyboard may include at least one of a switch, a key, a joystick, and a trackball. As another example, the keyboard may also be implemented in software, such as a graphic user interface. In this case, the keyboard may be displayed through a sub-display unit 162 or a main display unit 161. The foot switch or the foot pedal may be provided below the main body 100, and a manipulator may control operations of an ultrasound image generating apparatus using the foot pedal.

The display unit 160 may include the main display unit 161 and the sub-display unit 162.

The sub-display unit 162 may be provided in the main body 100. FIG. 1 illustrates a case in which the sub-display unit 162 is provided above the input unit 150. The sub-display unit 162 may display an application related to an operation of the ultrasound image generating apparatus. The sub-display unit 162 may display, for example, an instruction or a menu necessary for ultrasound diagnosis. This sub-display unit 162 may be implemented by, for example, a cathode ray tube (CRT), or a liquid crystal display (LCD).

The main display unit 161 may be provided in the main body 100. FIG. 1 illustrates a case in which the main display unit 161 is provided above the sub-display unit 162. The main display unit 161 may display an ultrasound image that is obtained in an ultrasound diagnosis process. This main display unit 161 may be implemented by the CRT or the LCD like the sub-display unit 162. FIG. 1 illustrates a case in which the main display unit 161 is combined with the main body 100. However, the main display unit 161 may also be detachable from the main body 100.

FIG. 1 illustrates a case in which both the main display unit 161 and the sub-display unit 162 are provided in the ultrasonic imaging apparatus. However, in some cases, the sub-display unit 162 may not be provided. In this case, the application, the menu, or the like displayed through the sub-display unit 162 may be displayed through the main display unit 161.

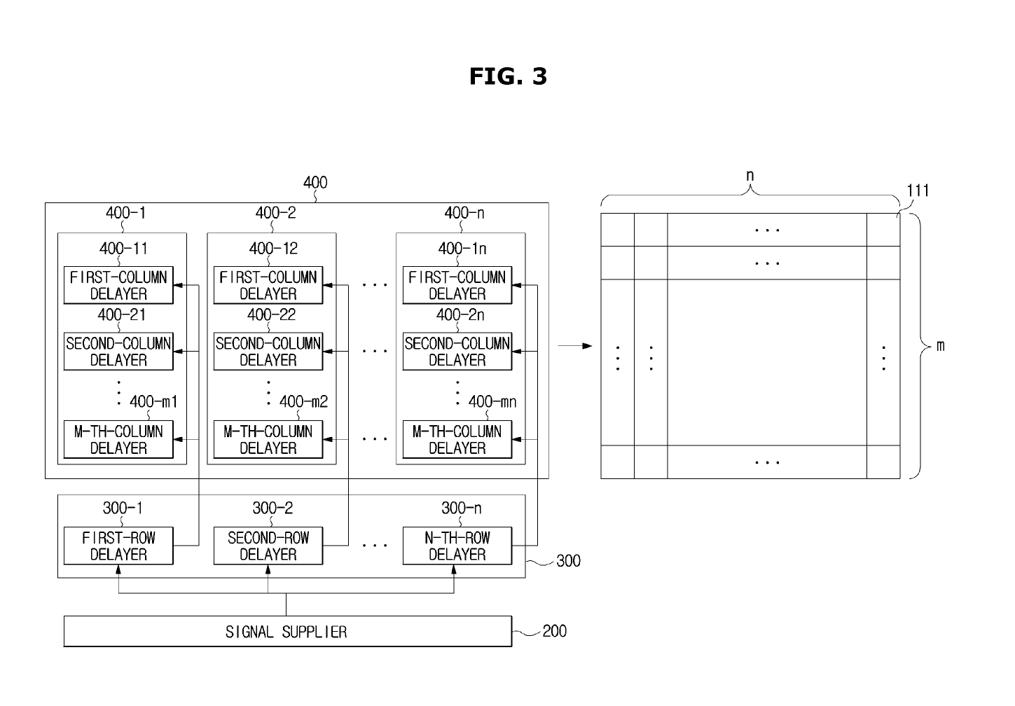

FIG. 3 is a block diagram of an ultrasonic apparatus according to an embodiment. In FIG. 3, a method will be described in which elements belonging to a row is set up as a sub-array.

The ultrasonic apparatus according to the embodiment may include a 2D array transducer 111 in which a plurality of elements are arranged so as to have a plurality of rows and columns; a signal supplier 200 configured to supply ultrasonic signals to the 2D array transducer; a row delayer 300 configured to primarily delay the ultrasonic signal to correspond to each of the plurality of rows; and a column delayer 400 configured to secondarily delay the first delayed ultrasonic signal to correspond to each of the plurality of elements.

The 2D array transducer 111 refers to the transducer of the 2D array probe 110 as described above with reference to FIG. 2

Each transducer element in the 2D array transducer 111 may be a magnetostrictive ultrasonic transducer using a magnetostrictive effect of a magnetic material, a piezoelectric ultrasonic transducer using a piezoelectric effect of a piezoelectric material, or a capacitive micro-machined ultrasonic transducer (abbreviated as cMUT hereinafter) transmitting and receiving ultrasonic waves by using vibrations of several hundreds or thousands of micro-machined thin films.

The 2D array transducer 111 has a larger number of elements than a 1D array transducer. Therefore, when all of the elements of the 2D array transducer 111 are set as channels, it is difficult to perform beamforming for all channels in the ultrasonic transmission/reception.

To solve this problem, a plurality of 1×m or n×1 sub-array transducers may be set up for the n×m 2D array transducer 111 to perform beamforming.

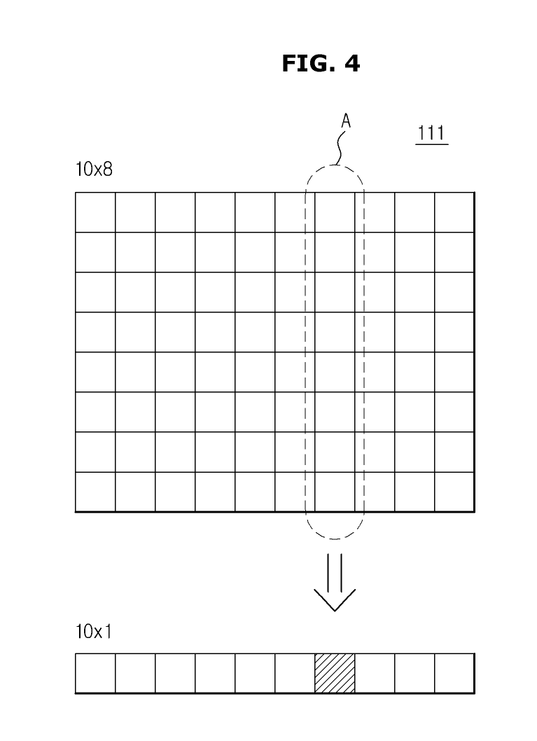

FIG. 4 is a diagram for explaining a method of setting up a sub-array transducer.

In the upper part of FIG. 4, an embodiment of an n×m 2D array transducer 111 is shown. To solve the problems that may occur when using the 2D array transducer 111, a plurality of sub-array transducers in the form of a 1D array transducer (i.e, a 1×m or n×1 sub-array transducer, may be set up.

As shown in the upper part of FIG. 4, elements existing in region A belong to the same row in the 2D array transducer 111. By setting up the elements belonging to the same row as a sub-array transducer, the 2D array transducer 111 may be recognized as a set of a plurality of 1D array transducers.

As shown in the lower part of FIG. 4, when elements belonging to the same row are set up as one sub-array transducer, the 2D array transducer 111 may be regarded as a 1D array transducer in which a plurality of element groups are arranged in one direction. For example, the elements in the region A shown in the upper part of FIG. 4 may be recognized as one element which is shaded in the lower part of FIG. 4.

Based on this, beamforming may be performed using each sub-array transducer in the same manner as beamforming using a 1D array transducer.

Setting up sub-array transducers for the 2D array transducer 111 means not physically separating the transducer elements, but controlling the ultrasonic signals to be supplied for each sub-array transducer.

Referring again to FIG. 3, the signal supplier 200 may supply the ultrasonic signal to the 2D array transducer 111. At this time, the ultrasonic signal supplied to the 2D array transducer 111 may be an Radio Frequency (RF) signal.

The transducer generates an ultrasonic wave by receiving an ultrasound, and transmits the ultrasonic wave to the object. Accordingly, the signal supplier 200 supplies the ultrasonic signals to the respective elements of the 2D array transducer 111, so that ultrasonic waves are generated in the respective elements.

The ultrasonic signal supplied from the signal supplier 200 may be properly delayed. When the ultrasonic signal is delayed according to the delay time corresponding to the element, beams are focused on a particular point.

In order to perform beamforming in each of all the elements of the 2D array transducer 111, the signal delay may be controlled for each element, so that the amount of computation may increase accordingly. If this is implemented in a circuit, the complexity of the circuit may also increase.

In order to solve this problem, as described above, signal delay may be controlled after dividing the n×m 2D array transducer 111 into a plurality of 1×m sub-array transducers.

The first delayer 300 may delay the ultrasound signal supplied from the signal supplier 200 to output a plurality of first delay signals corresponding to the number of the rows or the columns. This is to perform the signal delay by regarding each of the plurality of sub-array transducers as one element.

As described above, if each of the sub-array transducers is regarded as one element, the ultrasound signal may be delayed as if to perform beamforming on a 1D array transducer.

As shown in FIG. 3, the first delayer 300 may be provided to correspond to the rows of the 2D array transducer 111. Specifically, the first delayer 300 includes a first-row delayer 300-1 corresponding to the first row, a second-row delayer 300-2 corresponding to the second row, . . . , and an n-th-row delayer 300-n corresponding to the n-th row.

The first delayer 300 may delay the ultrasonic signal to have a delay time corresponding to the respective sub-array transducers. As a result, a number of first delay signals corresponding to the number of sub-array transducers may be output.

The number corresponding to the number of sub-array transducers may be the number of sub-array transducers, that is, the number of rows of the 2D array transducer 111. For example, in the case of the 2D array transducer 111 arranged to have n rows as shown in FIG. 3, the first delayer 300 may output n first delay signals.

At this time, the first delayer 300 may delay the ultrasonic signal in the same manner as the beamforming using the 1D array transducer. Accordingly, the first delayer 300 may delay the ultrasound signal so that the plurality of first delay signals have different delay times.

The second delayer 400 may delay one of the first delay signals output from the first delayer 300 to output a plurality of second delay signals corresponding to the other one of the rows or the columns. In FIG. 3, the second delayer 400 delays one of the first delay signals to output a plurality of second delay signals corresponding to the number of columns of the 2D array transducer 111.

A plurality of second delayer 400 may be provided to correspond to the respective sub-array transducers.

The second delayer 400 may be configured to correspond to elements belonging to each sub-array transducer. More specifically, the second delayer 400 may include first-column delayers 400-11, 400-12, . . . , and 400-1n corresponding to the elements located in the first column of the sub-array transducers, second-column delayers 400-21, 400-22, . . . , 400-2n corresponding to the elements located in the second column of the sub-array transducers, . . . , and m-th-column delayers 400-m1, 400-m2, . . . , and 400-mn corresponding to the elements located in the m-th column of the sub-array transducers.

The second delayer 400 may delay the ultrasound signal to have a delay time corresponding to each column of the sub-array transducers. As a result, a number of second delay signals corresponding to the number of columns of the sub-array transducer may be outputted.

The number corresponding to the number of columns of the sub-array transducer may be the number of elements belonging to one sub array transducer. For example, in the case of the 2D array transducer 111 arranged to have m columns as shown in FIG. 3, the second delayer 400 may output m second delay signals.

In other words, like the first delayer 300 delaying one ultrasonic signal to output n first delay signals corresponding to the number of rows, each second delayer 400 delays a first delay signal to output m second delay signals corresponding to the number of columns.

At this time, the second delayer 400 may delay the first delay signal in the same way in which Beamforming is performed using the 1D array transducer. Accordingly, the second delayer 400 may delay the first delay signal so that the plurality of second delay signals have different delay times.

The second delayer 400 may transmit the plurality of output second delay signals to the 2D array transducer 111. In particular, since each of the plurality of sub-array transducers may correspond to each of the plurality of first delay signals, the plurality of second delay signals output by delaying the same first delay signal may be transmitted to the sub-array transducer corresponding to the first delay signal.

Specifically, each of the plurality of first delay signals determines a row of the 2D array transducer 111 elements, and each of the second delay signals determines a column of the 2D array transducer 111 elements. Based on this, the second delay signal output by the second delayer 400 may be transmitted to the corresponding element.

Unlike FIG. 3, the sub-array transducer may be set up with a set of elements belonging to the same column. In this case, beamforming may also be performed in a manner similar to that of FIG. 3.

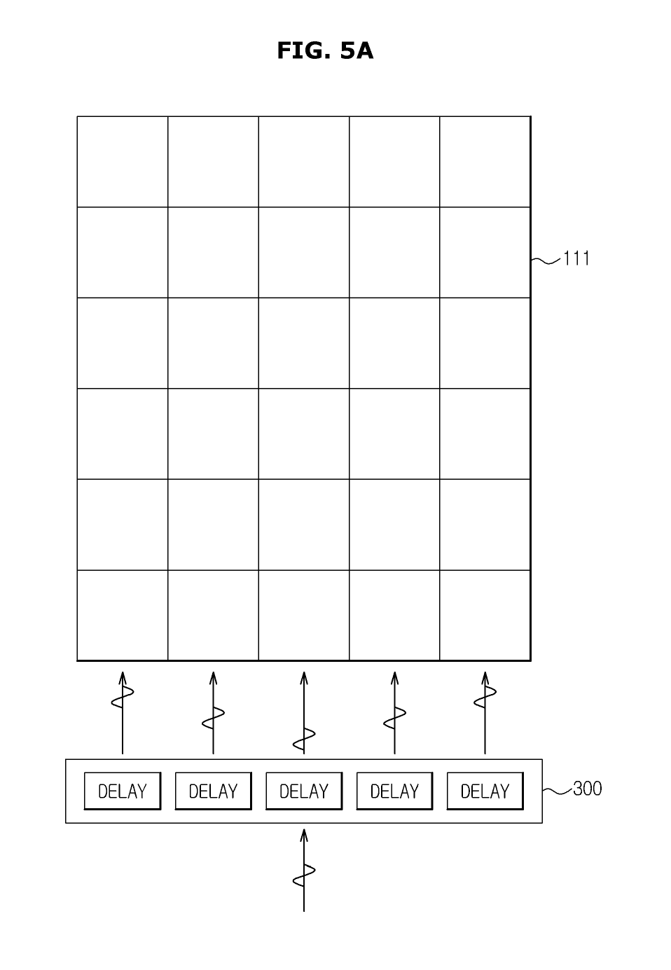

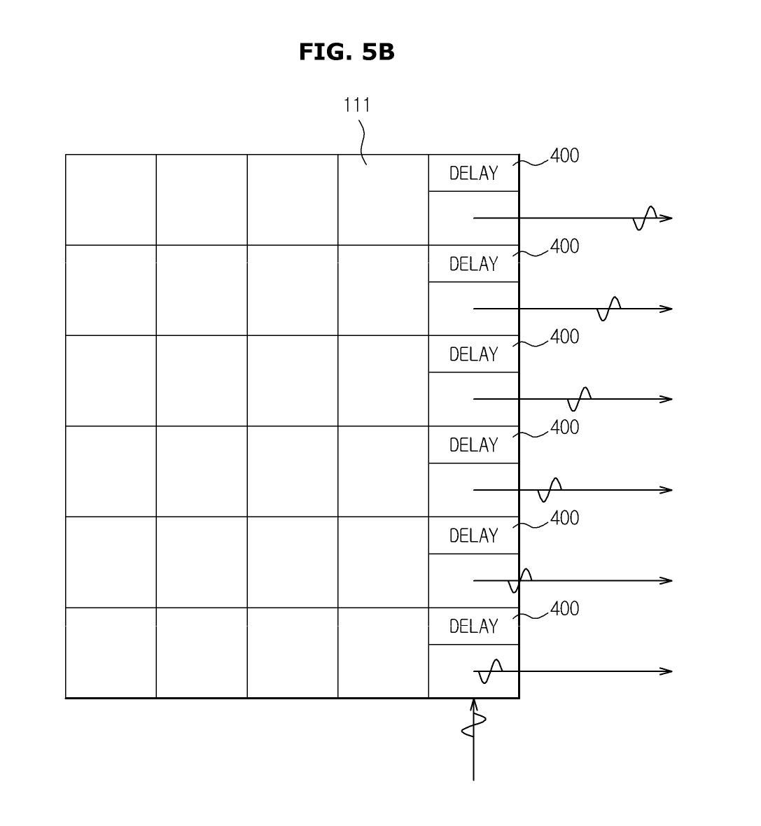

FIGS. 5A and 5B are views for explaining a method of setting up a sub-array transducer to perform beamforming in ultrasonic transmission.

As shown in FIG. 5A, one ultrasonic signal may be supplied. The supplied ultrasound signal is not directly transmitted to the 2D array transducer 111, but delayed twice.

The first delayer 300 delays the ultrasonic signal. Specifically, the ultrasonic signal is delayed as many delay times as the number of rows of the 2D array transducer 111.

As a result, a number of first delay signals corresponding to the number of rows of the 2D array transducer 111 are output. FIG. 5A, five first delay signals are output. At this time, the plurality of first delay signals may have different delay times.

The first delay signals output in this way may correspond to the rows of the 2D array transducer 111. That is, the first delay signals having different delay times affect no other than elements belonging to the corresponding row.

Referring to FIG. 5B, each of the output first delay signals may be delayed by the second delayer 400 to correspond to each element belonging to the corresponding row. FIG. 5B illustrates just a case where one first delay signal is delayed, for convenience of explanation.

The first delay signal is delayed by the delay time corresponding to each element belonging to the same row. As a result, a number of second delay signals corresponding to the number of elements belonging to the same row may be output from one first delay signal. In the case of FIG. 5B, there may be six second delay signals output. At this time, the plurality of second delay signals delayed from the same first delay signal may have different delay times.

As shown in FIGS. 5A and 5B, the sub-array transducer can be set up with a set of elements belonging to the same row. It is needless to say that it may also be set up with a set of elements belonging to the same column.

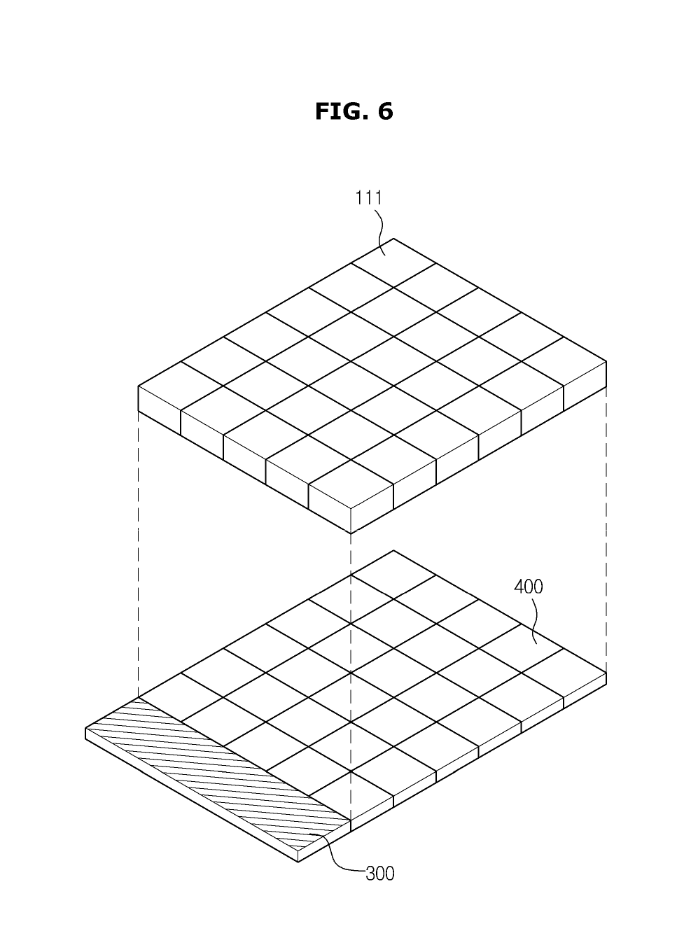

FIG. 6 is a view for explaining a structure in which a 2D array transducer, a first delayer and a second delayer are arranged. It will be described on assumption that the first delayer 300 and the second delayer 400 are implemented on a single circuit board.

In order to perform beamforming by controlling each element of the 2D array transducer 111, the ultrasonic signal may be delayed with a delay time corresponding to each of the plurality of elements. For this purpose, a configuration for transmitting ultrasonic signals having different delay time to the respective elements may be provided for the respective elements.

However, in the case of setting up the sub-array transducer, the first delayer 300 may be provided on the outer side of the circuit board as shown in FIG. 6. Accordingly, a delay may be primarily performed by the first delayer 300 before the ultrasonic signal is transmitted to the second delayer 400 corresponding to each element.

The beamforming method for transmitting ultrasounds using the sub-array transducer has thus far been described. Hereinafter, a beam forming method for receiving ultrasounds using the sub-array transducer will be described.

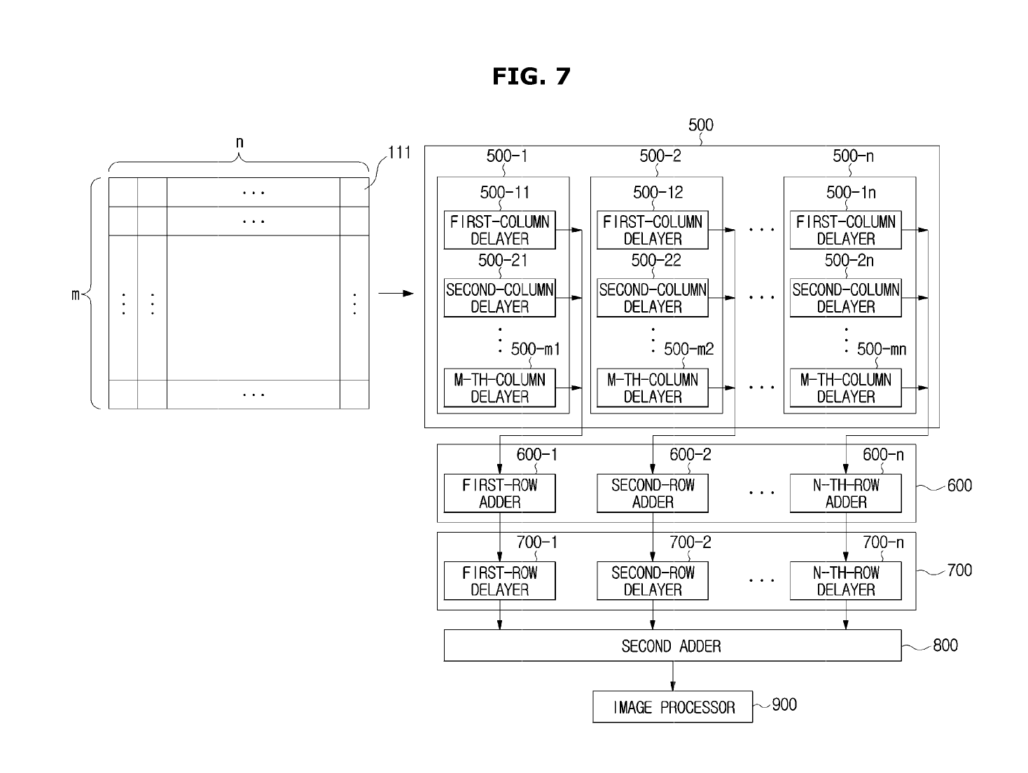

FIG. 7 is a block diagram illustrating another embodiment of the ultrasonic apparatus. In FIG. 7, a method of setting up elements as a sub-array will be described on the assumption that the elements belong to the same row.

According to another embodiment of the ultrasonic apparatus, the ultrasonic apparatus may include a 2D array transducer 111 configured to generate a plurality of ultrasonic echo signals by a plurality of elements arranged in a plurality of rows and columns; a third delayer 500 configured to delay the plurality of ultrasonic echo signals generated by the plurality of elements to output a plurality of third delay signals; a first adder 600 configured to combine third delay signals corresponding to either the same row or column among the plurality of third delay signals to output a plurality of first combined signals corresponding to the number of rows or columns; a fourth delayer 700 configured to delay the plurality of first combined signals to output a plurality of fourth delay signals; and a second adder 800 configured to combine the plurality of fourth delay signals to output a second combined signal.

The 2D array transducer 111 may receive ultrasounds to generate ultrasound waves, or receive reflected echo ultrasounds to output ultrasound echo signals. The 2D transducer 111 is the same 2D transducer 111 used for ultrasound transmission, so the detailed description will be omitted.

At this time, the ultrasonic echo signal may be an RF signal.

The 2D array transducer 111 includes a plurality of elements arranged in two dimensions, and the plurality of elements outputs a plurality of echo ultrasound signals. As in transmission of ultrasonic waves, if all the elements of the 2D array transducer 111 are used as channels for receiving echo ultrasonic waves, the subsequent signal processing process becomes complicated.

To solve this problem, the ultrasonic apparatus may be configured to delay the ultrasonic echo signal twice. This may mean that sub-array transducers are set up to perform beamforming.

To this end, the third delayer 500 may output a plurality of third delay signals by delaying the plurality of ultrasonic echo signals generated in the plurality of elements.

Specifically, the third delayer 500 may delay the plurality of ultrasonic echo signals according to the configuration of the sub-array. First, a plurality of elements belonging to the same row may be set up as one sub-array transducer. With the configuration, the third delayer 500 may group the plurality of ultrasonic echo signals generated by the same sub-array transducer into the same group. Finally, the plurality of ultrasonic echo signals belonging to the same group may be delayed. Delaying the plurality of ultrasonic echo signals in the unit of a group is similar to delaying the plurality of ultrasonic echo signals generated by one sub-array transducer, and therefore may be similar to a method of delaying the ultrasonic echo signal using a 1D array transducer is.

Referring to FIG. 7, there may be a plurality of third delayers 500 provided to correspond to the set up sub-array transducers. In particular, the third delayer 500 may be provided in the same number as the number of sub-array transducers, that is, the number of rows.

The third delayer 500 may delay each of the plurality of ultrasonic echo signals generated from the same sub-array transducer. To this end, the third delayer 500 may include first-column delayers 500-11, 500-12, . . . , and 500-1n corresponding to the elements located in the first column among the elements belonging to the same sub-array transducer, second-column delayers 500-21, 500-22, . . . , 500-2n corresponding to the elements located in the second column among the elements belonging to the same sub-array transducer, . . . and m-th-column delayers 500-m1, 500-m2, . . . , and 500-mn corresponding to the elements located in the m-th column among the elements belonging to the same sub-array transducer.

With the implementation, the third delayer 500 may delay a plurality of ultrasound echo signals in the unit of a group and output a number of third delay signals corresponding to the number of columns in each group. For example, in the case of FIG. 7, m third delay signals may be output for each group.

At this time, the m third delay signals output from each group may have different delay times.

When a plurality of third delay signals are output for each group, the first adder 600 may combine the third delay signals corresponding to either the same row or column among the plurality of third delay signals, and output a plurality of first combined signals corresponding to the number of rows or columns.

For example, in the case of FIG. 7, the first adder 600 may combine the third delay signals corresponding to the same row among the plurality of third delay signals, and output a plurality of first combined signals corresponding to the number of rows.

Specifically, the first adder 600 may combine the plurality of third delay signals output from the same group. That is, m third delay signals output from the same group corresponding to the same row may be combined.

As a result, the first adder 600 may output first combined signals. The first adder 600 may output a single first combined signal by combining a plurality of third delay signals output from the same group. At this time, since the first combined signal may be output for each group the first adder 600 may output a number of first combined signals corresponding to the number of groups, that is, the number of rows.

When the plurality of first combined signals are output, the fourth delayer 700 may output a plurality of fourth delay signals by delaying the plurality of combined signals.

Since the first adder 600 outputs the first combined signals corresponding to the respective sub-array transducers, the fourth delayer 700 takes each sub-array transducer for each element and delays the plurality of combined signals.

The fourth delayer 700 may delay the respective first combined signal with delay times corresponding to the respective sub-array transducers. At this time, the applied delay times may be different from each other.

As a result, the fourth delayer 700 may output a plurality of fourth delay signals delayed by the respective delay times corresponding to the sub-array transducers. Since each fourth delay signal corresponds to each sub-array transducer, the fourth delayer 700 may output the same number of fourth delay signals as the number of sub-array transducers, that is, the number of rows.

The second adder 800 combines the plurality of fourth delay signals output from the fourth delayer 700 and outputs one second combined signal. The second combined signal output may include information about a specific point inside the object.

The image processor 900 may generate an ultrasonic image for the target object based on the second combined signal.

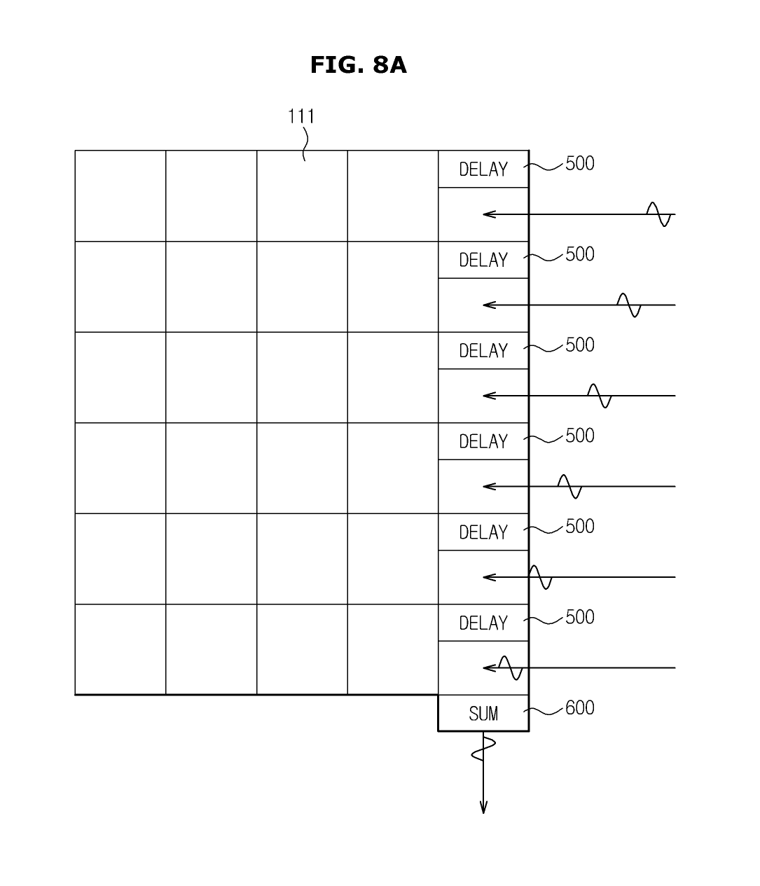

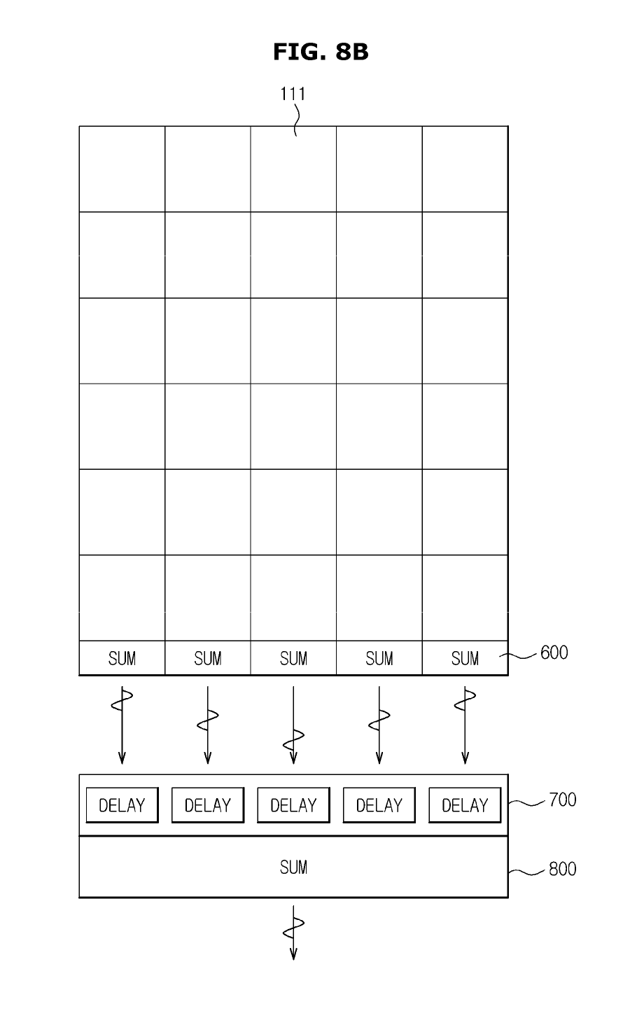

FIGS. 8A and 8B are views for explaining a method of setting up sub-array transducers to perform beamforming upon reception of ultrasound.

FIG. 8A illustrates operation of one sub-array transducer for convenience of explanation.

A set of elements belonging to the same row may be set up as a sub-array transducer. As shown in FIG. 8A, one sub-array transducer may receive an echo ultrasonic wave. Based on the received echo ultrasonic wave, each of the elements of one sub-array transducer may generate an ultrasonic echo signal.

Each of the ultrasonic echo signals may be delayed by the third delayer 500. The third delayer 500 may be provided for each sub-array transducer and may delay each ultrasonic echo signal with a delay time corresponding to each element.

As a result, the third delayer 500 may output a plurality of third delay signals. In the case of FIG. 8A, six third delay signals may be output.

Next, the first adder 600 may output one first combined signal by combining the six output third delay signals. The reason for combining the plurality of third delay signals together is to output the respective first combined signal corresponding to the respective sub-array transducer.

Referring to FIG. 8B, the respective first combined signals corresponding to the respective sub-array transducers are output. For example, five first combined signals may be output to correspond to five sub-array transducers.

By associating each sub-array transducer with each first combined signal, the sub-array transducer may be regarded as one element. In this way, a method of beamforming with a 1D array transducer may be similarly applied.

Specifically, the fourth delayer 700 may delay each of the first combined signals by the delay time corresponding to each sub-array transducer, and as a result, may output the fourth delay signal. In the case of FIG. 8B, five fourth delay signals may be output.

The plurality of fourth delay signals output in this manner may be combined by the second adder 800. As a result, the second adder 800 may output one second combined signal. Here, the second combined signal may include information about a specific point of the object, and may be used directly to generate an ultrasonic image.

As shown in FIGS. 8A and 8B, the sub-array transducer may be set up with a set of elements belonging to the same row. It is needless to say that it may also be set up with a set of elements belonging to the same column.

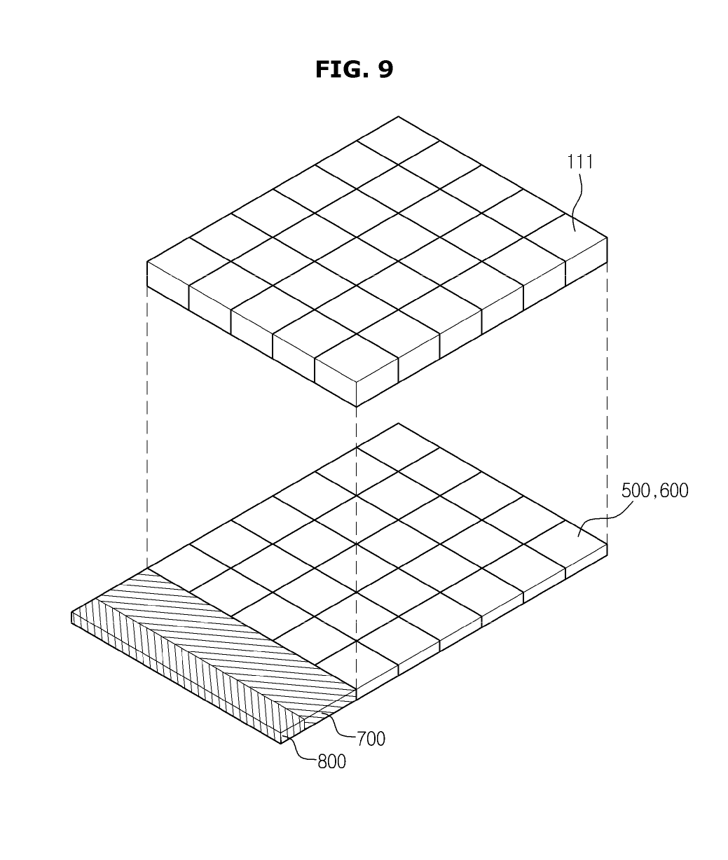

FIG. 9 is a view for explaining a structure in which the 2D array transducer, the third delayer, the fourth delayer, the first adder, and the second adder are arranged. Hereinafter, the case where the third delayer 500, the fourth delayer 700, the first adder 600, and the second adder 800 are implemented on one circuit board will be described.

In order to perform beamforming by controlling each element of the 2D array transducer 111, the ultrasonic echo signal may be delayed with a delay time corresponding to each of the plurality of elements. To this end, a configuration for delaying the ultrasonic echo signals generated by the respective elements may be provided for the respective elements.

However, in the case of beamforming echo signals by setting up the sub-array transducer, the fourth delayer 700 and the second adder 800 may be provided outside as shown in FIG. 9.

Unlike the third delayer 500 and the first adder 600 provided at positions corresponding to the 2D array transducer 111, the fourth delayer 700 is provided on the outer side of the circuit board. This is to delay the plurality of first combined signals generated according to the sub-array configuration.

The fourth delayer 700 and the second adder 800 are provided on the outer side of the circuit board to output a second combined signal by combining the plurality of fourth delay signals output by the fourth delayer.

In another embodiment of the ultrasonic apparatus, the first delayer 300 of FIG. 3 and the fourth delayer 700 of FIG. 7 may be implemented in a single configuration. In addition, the second delayer 400 of FIG. 3 and the third delayer 500 of FIG. 7 may be implemented in a single configuration.

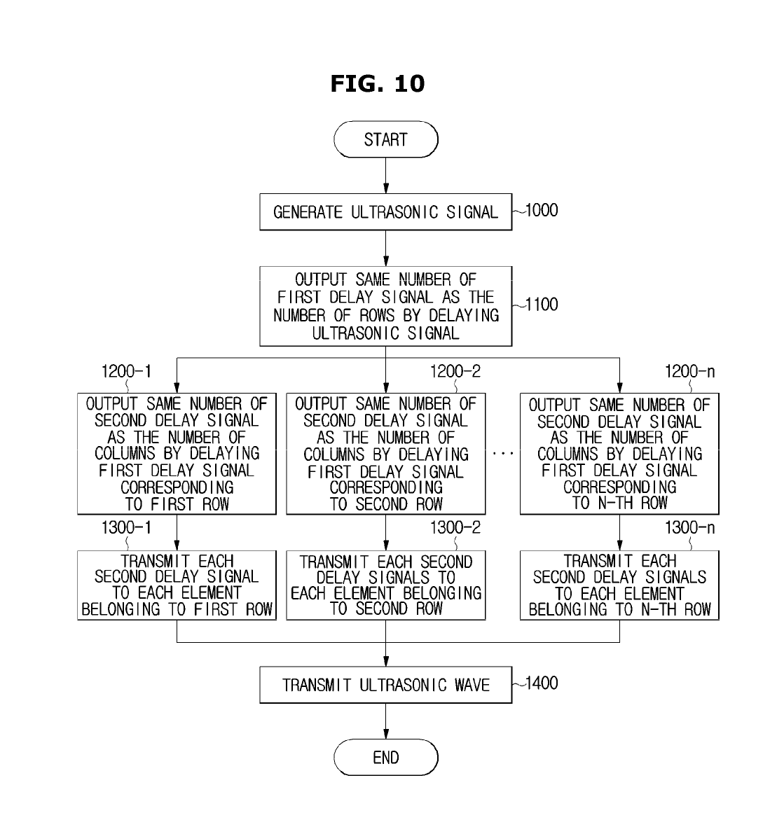

FIG. 10 is a flowchart according to an embodiment of a beamforming method of the ultrasonic apparatus. Specifically, it is assumed that ultrasonic waves are transmitted using the n×m 2D array transducer 111. It is also assumed that elements belonging to the same row are set up as a sub-array transducer.

First, an ultrasonic signal is generated in 1000. The ultrasonic signal may be generated by the signal supplier 200. The ultrasound signal generated at this time may be an RF signal.

The generated ultrasonic signal is delayed to output a number of first delay signals corresponding to the number of rows in 1100. By using the n×m 2D array transducer 111, there may be n first delay signals output.

By taking a sub-array transducer as an element, the n×m 2D array transducer 111 may be taken as an n×1 1D array transducer by setting up the elements belonging to the same row as a sub-array transducer. Therefore, the delay time as applied for beam forming of a 1D array transducer may be equally applied. As a result, the output first delay signals may have different delay times.

Each of the plurality of first delay signals output corresponds to each of the plurality of rows.

The first delay signal corresponding to the first row among the plurality of first delay signals is delayed to output a number of second delay signals corresponding to the number of columns, i.e., m second delay signals in 1200-1. The sub-array transducers are regarded as a 1D array transducer, based on which the first delay signal supplied may be delayed.

At this time, the plurality of second delay signals may have different delay times.

Each of the plurality of second delay signals output may be transmitted to each of the plurality of elements belonging to the first row, in 1300-1. In this way, the plurality of elements belonging to the first row have second delay signals with different delay times.

Likewise, the first delay signal corresponding to the second row may be delayed to output m second delay signals corresponding to the number of columns, in 1200-2, and each of the m second delay signals output may be transmitted to each of the elements belonging to the second row in 1300-1.

The same process may be repeated as many times as the number of rows. That is, the first delay signal corresponding to the n-th row may be delayed to output m second delay signals, and each of the m second delay signals may be output to each of the elements belonging to the n-th row, in 1300-n.

An ultrasonic wave is generated from each element having received the second delay signal, and the generated ultrasonic wave may be transmitted to the object in 1400.

In FIG. 10, a set of elements belonging to the same row is set up as a sub-array transducer. However, it is also possible to set up a set of elements belonging to the same column as a sub-array transducer. In this case, the beamforming may be performed in a manner similar to the beamforming method of FIG. 10.

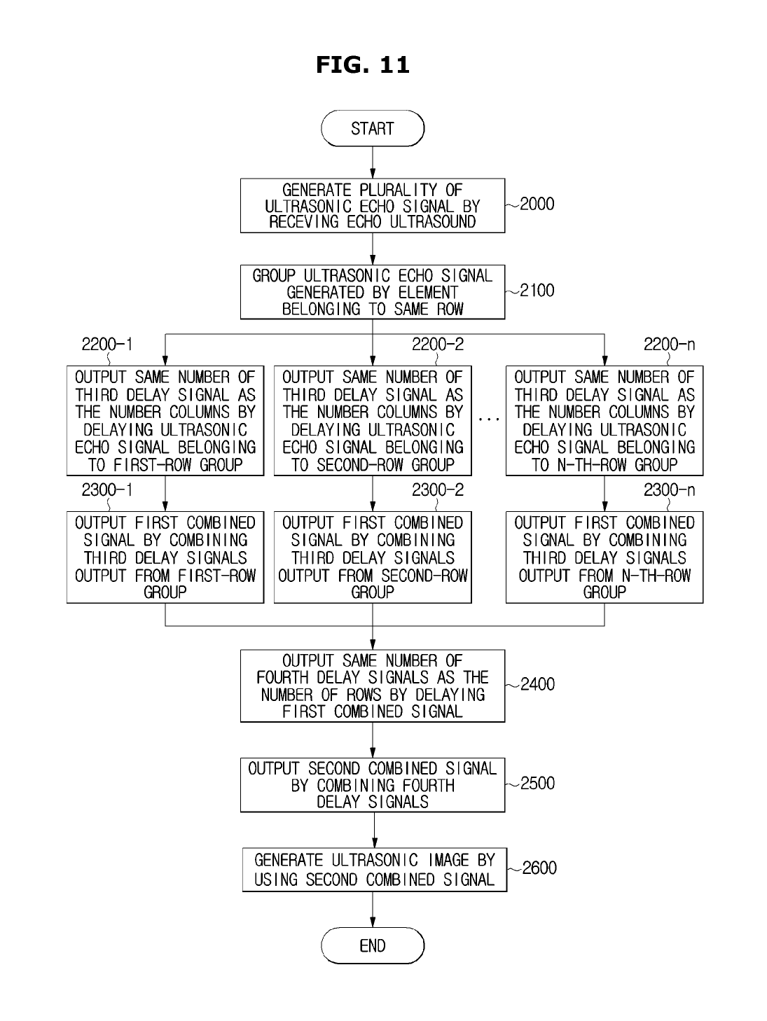

FIG. 11 is a flowchart according to another embodiment of the beam forming method of the ultrasonic apparatus. Specifically, it is assumed that an echo ultrasonic wave is received by using the n×m 2D array transducer 111. It is also assumed that elements belonging to the same row are set up as a sub-array transducer.

First, a plurality of ultrasonic echo signals may be generated by receiving echo ultrasonic waves through the 2D array transducer in 2000.

The ultrasonic echo signals generated from the elements belonging to the same row among the plurality of ultrasonic echo signals may be grouped in 2100. For example, a plurality of ultrasonic echo signals generated from the elements belonging to the first row may be grouped into a first-row group, a plurality of ultrasonic echo signals generated from the elements belonging to the second row may be grouped into a second-row group, . . . , a plurality of ultrasonic echo signals generated from the elements belonging to the n-th row may be grouped into an n-th-row group.

Next, the ultrasonic echo signals belonging to the first-row group may be delayed to output m third delay signals corresponding to the number of the columns in 2200-1. The ultrasonic echo signals belonging to the first-row group are regarded as ultrasonic echo signals generated from a 1D array transducer, and based on this, the plurality of ultrasonic echo signals are delayed.

At this time, the third delay signals may have different delay times.

The m third delay signals output from the first row group are combined to output a first combined signal in 2300-1. The output first combined signal may correspond to a sub-array transducer composed of elements belonging to the first row.

Likewise, ultrasonic echo signals belonging to the second-row group may be delayed to output m third delay signals corresponding to the number of the columns in 2200-2. The m third delay signals output from the second row group are combined to output another first combined signal, in 2300-2.

The same process may be repeated as many times as the number of rows. That is, ultrasonic echo signals belonging to the second-row group may be delayed to output m third delay signals corresponding to the number of the columns in 2200-n, and the output m third delay signals are combined to output another first combined signal in 2300-n.

As a result, n first combined signals may be output.

When the n first combined signals are output, the n first combined signals are delayed to output n fourth delay signals corresponding to the number of the rows, in 2400. At this time, each of the n first combined signals corresponds to each of the sub-array transducers. Therefore, if a sub-array transducer is regarded as an element, delaying the n first combined signals may be similar to delaying the ultrasonic echo signals acquired by the n×1 1D array transducer.

After the n fourth delayed signals are output, they may be combined to output a second combined signal in 2500. The second combined signal may include information about a specific point within the object. Therefore, an ultrasonic image may be generated based on the second combined signal in 2600.

In FIG. 11, a set of elements belonging to the same row is set up as a sub-array transducer. However, it is also possible to set up a set of elements belonging to the same column as a sub-array transducer. In this case, beamforming may be performed in a manner similar to the beamforming method of FIG. 11.

1. An ultrasonic apparatus comprising:
a 2D array transducer in which a plurality of elements are arranged in a plurality of rows and columns;
a signal supplier configured to supply an ultrasonic signal to the 2D array transducer;
a first delayer configured to delay the ultrasonic signal to output a plurality of first delay signals corresponding to any one of the number of rows and the number of columns; and
a second delayer configured to delay one of the plurality of first delay signals to output a plurality of second delay signals corresponding to the other one of the number of rows and the number of columns, and transmit the plurality of second delay signals to the 2D array transducer.

2. The ultrasonic apparatus according to claim 1, wherein the first delayer is configured to output the plurality of first delay signals, the number of which is equal to one of the number of rows and the number of columns, and
wherein the second delayer is configured to output a plurality of second delay signals, the number of which is equal to the other one of the number of rows and the number of columns.

3. The ultrasonic apparatus according to claim 1, wherein the first delayer is configured to delay the ultrasonic signal so that the plurality of first delay signals have different delay time.

4. (canceled)

5. The ultrasonic apparatus according to claim 1, wherein the second delayer is configured to delay one of the plurality of first delay signals so that the plurality of second delay signals have different delay time.

6. The ultrasonic apparatus according to claim 1, wherein the second delayer is configured to transmit each of the plurality of second delay signals to each element belonging to one of the rows or the columns corresponding to one of the plurality of first delay signals.

7. An ultrasonic apparatus comprising:
a 2D array transducer configured to generate a plurality of ultrasonic echo signals by a plurality of elements arranged in a plurality of columns and rows;
a third delayer configured to delay each of the plurality of ultrasonic echo signals generated in each of the plurality of elements, and output a plurality of third delay signals;
a first adder configured to combine the third delay signals corresponding to one of the plurality of third delay signals to output a plurality of first combined signals corresponding to one of the number of the rows and the number of the columns;
a fourth delayer configured to delay the plurality of first combined signals to output a plurality of fourth delay signals; and
a second adder configured to combine the plurality of fourth delay signals to output a second combined signal.

8. The ultrasonic apparatus according to claim 7, wherein the third delayer is configured to group the plurality of ultrasonic echo signals according to one of the row and the column to which the elements from which the plurality of ultrasonic echo signals are generated belongs, and output a plurality of third delay signals for each group generated by the grouping.

9. The ultrasonic apparatus according to claim 8, wherein the third delayer is configured to output a plurality of third delay signals for each group, the number of which corresponds to the other one of the number of rows and the number of columns.

10. The ultrasonic apparatus according to claim 8, wherein the third delayer is configured to output a plurality of third delay signals having different delay times from the same group, and
wherein the first adder is configured to combine the plurality of third delay signal generated from the same group to output one first combined signal.

11. (canceled)

12. The ultrasonic apparatus according to claim 7, wherein the fourth delayer is configured to delay each of the plurality of first combined signals so that each of the plurality of fourth delay signals has different delay time.

13. A beamforming method of an ultrasonic apparatus including a 2D array transducer in which a plurality of elements are arranged in a plurality of columns and rows, the method comprising:
generating an ultrasonic signal;
delaying the ultrasonic signal to output a plurality of first delay signals, the number of which corresponds to one of the number of the rows and the number of the columns;
delaying one of the plurality of first delay signals to output a plurality of second delay signals, the number of which corresponds to the other one of the number of the rows and the number of the columns; and
transmitting the plurality of second delay signals to the 2D array transducer.

14. The method according to claim 13, wherein outputting a plurality of first delay signals comprises outputting a plurality of first delay signals, the number of which is equal to any one of the number of rows and the number of columns, and
wherein outputting a plurality of second delay signals comprises outputting a plurality of second delay signals, the number of which is equal to the other one of the number of rows and the number of columns.

15. The method according to claim 13, wherein delaying the ultrasonic signal comprises delaying the ultrasonic signal so that each of the plurality of first delay signals have different delay time.

16. (canceled)

17. The method according to claim 13, wherein outputting the plurality of second delay signals comprises delaying one of the first delay signals so that each of the plurality of second delay signals have different delay time.

18. The method according to claim 13, wherein transmitting the plurality of second delay signals comprises transmitting each of the plurality of second delay signals to each element belonging to one of the row and the column corresponding to one of the plurality of first delay signals.

19. A beamforming method of an ultrasonic apparatus including a 2D array transducer configured to generate a plurality of ultrasonic echo signals by a plurality of elements arranged in a plurality of columns and rows, the method comprising:
delaying each of the plurality of ultrasonic echo signals generated by each of the plurality of elements to output a plurality of third delay signals;
combining the third delay signals corresponding to one of the same row and the same column among the plurality of third delay signals to output a plurality of first combined signals corresponding to one of the number of rows and the number of columns;
delaying the plurality of first combined signals to output a plurality of fourth delay signals; and
combining the plurality of fourth delay signals to output one second combined signal.

20. The method according to claim 19, wherein outputting a plurality of third delay signals comprises:
grouping the plurality of ultrasonic echo signals according to one of the row and the column to which the elements from which the plurality of ultrasonic echo signals are generated belongs; and
outputting a plurality of third delay signals for each group generated by the grouping.

21. The method according to claim 20, wherein outputting the plurality of third delay signals for each group comprises outputting a plurality of third delay signals for each group, the number of which corresponds to the other one of the number of rows and the number of columns.

22. The method according to claim 20, wherein outputting a plurality of third delay signals for each group comprises outputting a plurality of third delay signals having different delay times in the same group, and wherein outputting a plurality of fourth delay signals comprises combining the plurality of third delay signal generated from the same group to output one first combined signal.

23. (canceled)

24. The method according to claim 19, wherein delaying the plurality of first combined signals comprises delaying each of the plurality of first combined signals so that each of the plurality of fourth delay signals has different delay time.
